(19) United States
(12) Patent Application Publication
(10) Pub. No.: US 2007/0092459 A1
(43) Pub. Date: Apr. 26, 2007

(54) Cosmetic preparation with 1,2-alkanediol and triazines

(75) Inventors: Andreas Bleckmann, Ahrensburg (DE); Svea Behrens, Hamburg (DE); Andreas Clausen, Hamburg (DE); Uta Meiring, Hamburg (DE); Jens Nielsen, Henstedt-Ulzburg (DE)

Correspondence Address: GREENBLUM & BERNSTEIN, P.L.C., 1950 ROLAND CLARKE PLACE, RESTON, VA, 20191, US

(73) Assignee: Beiersdorf AG, Hamburg (DE)

(21) Appl. No.: 11/585,246

(22) Filed: Oct. 24, 2006

(30) Foreign Application Priority Data
Oct. 25, 2005 (DE) 10 2005 051 858.3

(51) Int. Cl. A61K 8/49

(52) U.S. Cl. 424/59

(57) ABSTRACT

A cosmetic preparation which comprises one or more 1,2-alkanediols and one or more UV light-protection filters from the group of triazine derivatives. This Abstract is not intended to define the invention disclosed in the specification, nor intended to limit the scope of the invention in any way.

### CROSS-REFERENCE TO RELATED APPLICATIONS

The present application claims priority under 35 U.S.C. § 119 of German Patent Application No. 10 2005 051 858.3, filed Oct. 25, 2005, the disclosure of which is expressly incorporated by reference herein in its entirety.

### BACKGROUND OF THE INVENTION

1. Field of the Invention

The present invention relates to a cosmetic preparation which comprises one or more 1,2-alkanediols and one or more UV light-protection filters from the group of triazine derivatives.

2. Discussion of Background Information

The trend away from genteel pallor towards “healthy, athletic tanned skin” has been unbroken for years. In order to attain this, people expose their skin to solar radiation since this brings about pigment formation in the sense of melanine formation. However, the ultraviolet radiation of sunlight also has a harmful effect on the skin. Besides acute damage (sunburn), long-term damage occurs, such as suffering from an increased risk of skin cancer in cases of excessive irradiation with light from the UVB region (wavelength: 280-320 nm). Moreover, the excessive effect of UVB and UVA radiation (wavelength: 320-400 nm) leads to a weakening of the elastic and collagenous fibers of connective tissue. This leads to numerous phototoxic and photoallergic reactions and results in premature skin ageing.

To protect the skin, a number of light-protection filters have therefore been developed which can be used in cosmetic preparations. These UVA and UVB filters are summarized in most industrialized countries in the form of positive lists such as Annex 7 of the Cosmetics Directive.

One important group of UV light-protection filters is based on the structural element of triazine

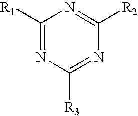

### Basic Structure of Triazine

UV light-protection filters that have the skeletal structure of triazine (triazine derivatives) have the disadvantage that they are only poorly soluble in cosmetic preparations. This fact leads to problems in particular when preparations are to be produced with a high light-protection factor, since in these cases only insufficient quantities of triazine derivatives can be incorporated into the preparation.

It would be desirable to have available preparations into which larger amounts of triazine derivatives can be incorporated. In particular, it would be advantageous to develop preparations with a high light-protection factor. Due to the absorption spectrum of triazine derivatives adapted to the protective needs of human skin, these preparations are to have a particularly high content of this UV filter class.

### SUMMARY OF THE INVENTION

The present invention provides a cosmetic preparation which comprises (a) one or more 1,2-alkanediols and (b) one or more UV light-protection filters from the group of triazine derivatives.

In one aspect, the preparation may comprise (a) in a total concentration of from about 0.3% to about 3% by weight, e.g., from about 0.5% to about 2% by weight, based on the total weight of the preparation.

In another aspect, the preparation may comprise (b) in a total concentration of from about 0.3% to about 3% by weight, e.g., from about 0.5% to about 2% by weight, based on the total weight of the preparation.

In another aspect, the weight ratio (a): (b) may be from about 2:1 to about 1:2, e.g., about 1:1.

In yet another aspect, (a) may comprise 1,2-hexanediol and/or (b) may comprise one or more of 2,4-bis{[4-(2-ethylhexyloxy)-2-hydroxy]phenyl}-6-(4-methoxyphenyl)-1,3,5-triazine, dioctylbutylamidotriazone, 2,4-bis-[5-1 (dimethylpropyl)benzoxazol-2-yl-(4-phenyl)-imino]-6-(2-ethylhexyl)-imino-1,3,5-triazine, tris(2-ethylhexyl) 4,4′,4″-(1,3,5-triazine-2,4,6-triyltriimino)trisbenzoate and 2,4,6-tribiphenyl-4-yl-1,3,5-triazine, e.g., one or more of 2,4-bis{[4-(2-ethylhexyloxy)-2-hydroxy]phenyl}-6-(4-methoxyphenyl)-1,3,5-triazine, dioctylbutylamidotriazone, 2,4-bis-[5-1 (dimethylpropyl)benzoxazol-2-yl-(4-phenyl)-imino]-6-(2-ethylhexyl)-imino-1,3,5-triazine and 2,4,6-tribiphenyl-4-yl-1,3,5-triazine.

In a still further aspect, the preparation of the present invention may comprise one or more additional UV light-protection filters which are different from a triazine derivative such as, for example, one or more of phenylene-1,4-bis(2-benzimidazyl)-3,3′-5,5′-tetrasulfonic acid salts, 2-phenylbenzimidazole-5-sulfonic acid salts, 1,4-di(2-oxo-10-sulfo-3-bornylidenemethyl)-benzene and salts thereof, 4-(2-oxo-3-bornylidenemethyl)benzenesulfonic acid salts, 2-methyl-5-(2-oxo-3-bornylidenemethyl)sulfonic acid salts, 2,2′-methylenebis[6-(2H-benzotriazol-2-yl)-4-(1,1,3,3-tetramethylbutyl)phenol], 2-(2H-benzotriazol-2-yl)-4-methyl-6-[2-methyl-3-[1,3,3,3-tetramethyl-1-[(trimethylsilyl)oxy]-disiloxanyl]-propyl]-phenol, 3-(4-metylbenzylidene)camphor, 3-benzylidene camphor, (2-ethylhexyl) 4-(dimethylamino)benzoate, amyl 4-(dimethylamino)benzoate; di(2-ethylhexyl) 4-methoxybenzalmalonate; dioctylbutylamidotriazone, (2-ethylhexyl) 4-methoxycinnamate, isopentyl 4-methoxycinnamate; 2-hydroxy-4-methoxybenzophenone, 2-hydroxy-4-methoxy-4′-methylbenzophenone, 2,2′-dihydroxy-4-methoxy-benzophenone, hexyl 2-(4′-diethylamino-2′-hydroxybenzoyl)-benzoate, 4-(tert.-butyl)-4′-methoxydibenzoylmethane, homomenthyl salicylate, 2-ethylhexyl-2-hydroxybenzoate, 2-ethylhexyl-2-cyano-3,3-diphenylacrylate, 3-(4-(2,2-bis-ethoxycarbonylvinyl)-phenoxy)propenyl)-methoxysiloxane/-dimethyl siloxane-copolymer, titanium dioxide and zinc oxide.

In another aspect, the preparation may further comprise tocopheryl acetate and/or glycerol.

The present invention also provides a cosmetic preparation which comprises (a) from about 0.5% to about 2% by weight of one or more 1,2-alkanediols which comprise 1,2-hexanediol and (b) from about 0.5% to about 2% by weight of one or more UV light-protection filters from the group of triazine derivatives.

In one aspect of this preparation, the weight ratio (a): (b) may be from about 2:1 to about 1:2.

In another aspect, (b) may comprise one or more of 2,4-bis{[4-(2-ethylhexyloxy)-2-hydroxy]phenyl}-6-(4-methoxyphenyl)-1,3,5-triazine, dioctylbutylamidotriazone, 2,4-bis-[5-1 (dimethylpropyl)benzoxazol-2-yl-(4-phenyl)-imino]-6-(2-ethylhexyl)-imino-1,3,5-triazine and 2,4,6-tribiphenyl-4-yl-1,3,5-triazine.

In a still further aspect, the preparation may comprise one or more additional UV light-protection filters which are different from a triazine derivative.

The present invention also provides an emulsion which comprises a preparation of the present invention as set forth above, including the various aspects thereof.

The present invention also provides a method of improving the solubility of a UV light-protection filter which is a triazine derivative. The method comprises combining the UV light-protection filter with one or more 1,2-alkanediols.

### DETAILED DESCRIPTION OF THE PRESENT INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description making apparent to those skilled in the art how the several forms of the present invention may be embodied in practice.

Although EP 1 238 651 and EP 1 078 638 describe preparations with light-protection filters and alkanediols, these documents were not able to show the way to the present invention.

It is advantageous if the preparation according to the present invention comprises one or more 1,2-alkanediols in a total concentration of from about 0.3% to about 3% by weight, based on the total weight of the preparation.

It is further preferred if the preparation according to the present invention comprises one or more 1,2-alkanediols in a total concentration of from about 0.5% to about 2% by weight, based on the total weight of the preparation.

It also is advantageous for the purposes of the present invention if the preparation comprises one or more UV light-protection filters selected from triazine derivatives in a total concentration of from about 0.3% to about 3% by weight, based on the total weight of the preparation.

It is preferred within the scope of the present invention if the preparation comprises one or more UV light-protection filters from the group of triazine derivatives in a total concentration of from about 0.5% to about 2.0% by weight, based on the total weight of the preparation.

Advantageous embodiments within the scope of the present invention are exhibit a weight ratio of the total amount of 1,2-alkane diols to the total amount of UV light-protection filters from the group of triazine derivatives in the preparations from about 2:1 to about 1:2, particularly preferably about 1:1.

It is particularly preferred for the purposes of the present invention if the one or more 1,2-alkanediols comprise 1,2-hexanediol. Other examples of suitable 1,2-alkanediols are those which comprise from about 2 to about 10 carbon atoms, e.g., from about 3 to about 8 carbon atoms, or from about 4 to about 7 carbon atoms, e.g., about 5 carbon atoms or about 6 carbon atoms.

It also is advantageous according to the present invention if one or more of the following compounds are chosen as UV light-protection filter from the group of triazine derivatives:
2,4-bis{[4-(2-ethylhexyloxy)-2-hydroxy]phenyl}-6-(4-methoxyphenyl)-1,3,5-triazine (INCI: Aniso Triazine, which is available under the trade name Tinosorb S),
dioctylbutylamidotriazone (INCI: Dioctylbutamidotriazone),
2,4-bis-[5-1 (dimethylpropyl)benzoxazol-2-yl-(4-phenyl)-imino]-6-(2-ethylhexyl)-imino-1,3,5-triazine (CAS No. 288254-16-0), which is available from 3V Sigma under the trade name Uvasorb® K2A).
4,4′,4″-(1,3,5-triazine-2,4,6-triyltriimino)tris-benzoic acid-tris(2-ethyl-hexylester) (also: 2,4,6-tris[anilino(p-carbo-2′-ethyl-1′-hexyloxy)]-1,3,5-triazine (INCI: Octyl Triazone), which is sold by BASF Aktiengesellschaft under the trade name UVINUL® T 150;
2,4,6-tribiphenyl-4-yl-1,3,5-triazine.

Furthermore, the preparation according to the present invention may advantageously comprise other UV light-protection filters. The UV light-protection filters approved by the Cosmetics Directive are particularly advantageous. Additional filters of this type can be advantageously contained in the preparation according to the present invention in concentrations of from about 0.01% to about 30% by weight based on the total weight of the preparation. It is preferred according to the present invention if one or more further UV light-protection filters are chosen from phenylene-1,4-bis(2-benzimidazyl)-3,3′-5,5′-tetrasulfonic acid salts, 2-phenylbenzimidazole-5-sulfonic acid salts, 1,4-di(2-oxo-1 0-sulfo-3-bornylidenemethyl)-benzene and salts thereof, 4-(2-oxo-3-bornylidenemethyl)benzene sulfonic acid salts, 2-methyl-5-(2-oxo-3-bornylidenemethyl)sulfonic acid salts, 2,2′-methylene-bis[6-(2H-benzotriazol-2-yl)-4-(1,1,3,3-tetramethylbutyl)phenol], 2-(2H-benzotriazol-2-yl)-4-methyl-6-[2-methyl-3-[1,3,3,3-tetramethyl-1-[(trimethylsilyl)oxy]disiloxanyl]propyl]-phenol, 3-(4-methylbenzylidene)camphor, 3-benzylidene camphor, (2-ethylhexyl) 4-(dimethylamino)benzoate, amyl 4-(dimethylamino)benzoate; di(2-ethylhexyl) 4-methoxybenzalmalonate; dioctyl butylamidotriazone, (2-ethylhexyl) 4-methoxycinnamate, isopentyl 4-methoxycinnamate; 2-hydroxy-4-methoxy-benzophenone, 2-hydroxy-4-methoxy-4′-methylbenzophenone, 2,2′-dihydroxy-4-methoxybenzophenone, hexyl 2-(4′-diethylamino-2′-hydroxybenzoyl)-benzoate, 4-(tert.-butyl)-4′-methoxydibenzoylmethane, homomenthyl salicylate, 2-ethylhexyl-2-hydroxybenzoate, 2-ethylhexyl-2-cyano-3,3-diphenylacrylate, 3-(4-(2,2-bis-ethoxycarbonylvinyl)-phenoxy)propenyl)-methoxysiloxane/dimethyl siloxane-copolymer, titanium dioxide, zinc oxide.

It is preferred for the preparation according to the present invention to be free from 3-(4-methylbenzylidene)camphor.

Cosmetic preparations according to the present invention can be present in different forms. They can therefore be present in the form of, e.g., a solution, a nonaqueous preparation, an emulsion or microemulsion of the water-in-oil type (W/O) or the oil-in-water type (O/W), a multiple emulsion, e.g., of the water in oil-in-water (W/O/W) type, a gel, a solid stick, an ointment, a foam or an aerosol.

It is preferred for the cosmetic preparation to be present in the form of an emulsion and particularly preferred for the preparation to be present in the form of an O/W emulsion. It is preferred according to the present invention for emulsions of this type to be free from sodium dihydroxycetyl phosphate, e.g., Dragophos S).

It is advantageous for the preparation according to the present invention to contain tocopheryl acetate. Tocopheryl acetate is advantageously contained in the preparation (preferably emulsion) in a concentration of from about 0.1% to about 1.0% by weight and preferably in a concentration of from about 0.3% to about 0.7% by weight, based on the total weight of the preparation.

Further, the preparation according to the present invention can advantageously be present as an ointment, a cream or a lotion (possibly sprayable). The preparation (e.g., emulsion) according to the present invention can also be used advantageously as a spray or impregnation medium for a bandage or a wipe.

The aqueous phase of a preparation according to the present invention can advantageously contain conventional cosmetic auxiliaries, such as, e.g., alcohols, in particular those with a low carbon number, preferably ethanol and/or isopropanol, diols or polyols of low carbon number, and ethers thereof, preferably ethanol, isopropanol, propylene glycol, glycerin, ethylene glycol, ethylene glycol monoethyl or monobutyl ether, propylene glycol monomethyl, monoethyl or monobutyl ether, diethylene glycol monomethyl or monoethyl ether and analogous products, polymers, foam stabilizers, electrolytes, self-tanning agents (e.g., dihydroxyacetone), insect repellents and in particular one or more thickeners, which can be advantageously chosen from silicon dioxide, aluminum silicates, polysaccharides or derivatives thereof, e.g., hyaluronic acid, xanthan gum, hydroxypropylmethylcellulose, particularly advantageously from the group of polyacrylates, preferably a polyacrylate from the group of so-called carbopols, e.g., carbopol grades 980, 981, 1382, 2984, 5984, in each case individually or in combination.

The oil phase of a preparation according to the present invention can contain all the conventional constituents of oil, fat and wax components used in cosmetics.

The preparation according to the invention can advantageously further comprise cosmetic active ingredients and care substances, e.g., preservatives or preservative auxiliaries authorized under the Cosmetics Directive. As further care substances, in particular niacinamide, panthenol, aloe vera, hammamelis extract, polidocanol, vitamin E, vitamin A, vitamin A derivatives, vitamin C, vitamin C derivatives, coenzyme Q10, creatine, taurine, alpha-glycosylrutin can be used, Ingredients of this type can be advantageously contained in the preparation according to the present invention in concentrations of from about 0.01% to about 30% by weight, e.g., from about 0.1% to about 30% by weight, based on the total weight of the preparation.

It also is preferred for the preparations according to the present invention to be free from iodopropynyl butylcarbamate.

It is particularly preferred for the preparations of the present invention to comprise as further constituents alpha-hydroxy acids and/or salts thereof. Lactic acid/lactate or citric acid/citrate are particularly preferred and may be present in a concentration of, for example, from about 0.01% to about 5% by weight based on the total weight of the preparation.

The preparation according to the present invention may also comprise other ingredients such as, e.g., perfumes in any desired concentration and quantity.

Unless stated otherwise, the numbers in the following Examples refer to % by weight.

### EXAMPLES

### (O/W Emulsions)

| | Example | | | | | | | |
|---|---|---|---|---|---|---|---|---|
| | 1 | 2 | 3 | 4 | 5 | 6 | 7 | 8 |
| Glyceryl stearate citrate | | 1.0 | | 2.0 | | | | 2.0 |
| PEG-40 stearate | 1.0 | | | | | 1.5 | | |
| Polyglyceryl | | | 2.0 | | 3.0 | | 1.5 | 0.5 |
| methylglucose distearate | | | | | | | | |
| Glyceryl stearate | 1.5 | | | | | 1.5 | | |
| Cetyl alcohol | | 0.5 | | 2.0 | 1.5 | 0.75 | 1.0 | |
| Stearyl alcohol | | 0.5 | 0.5 | | | 0.75 | | |
| Cetearyl alcohol | 2.0 | | | | | | | 1.5 |
| Caprylic/capric | 5.0 | 4.0 | | 5.0 | | 6.0 | 3.0 | 0.5 |
| triglyceride | | | | | | | | |
| Ethylhexylcocoate | | | 2.0 | | | | 1.0 | |
| Octyldodecanol | | 1.0 | 3.0 | | 5.0 | | 2.0 | |
| Mineral oil | | | 2.0 | | | | | |
| Hydrogenated | 1.0 | | | | | | | |
| polyisobutene | | | | | | | | |
| Polydecene | | | | 2.0 | | | | |
| Cyclomethicone | 2.0 | | | | | 3.0 | | |
| Dimethicone | 1.0 | | | | | | | 1.5 |
| Phenyltrimethicone | | | | | | 1.0 | | |
| Dicaprylyl carbonate | | 2.0 | | | 2.0 | | | 3.5 |
| Natural oils (such as, | 1.5 | | 3.0 | 0.5 | 1.0 | | 2.0 | 2.5 |
| e.g., jojoba oil/sunflower | | | | | | | | |
| oil) | | | | | | | | |
| 1,2-Hexanediol | 0.5 | 0.75 | 3.0 | 0.3 | 2.0 | 1.5 | 1.0 | 0.75 |
| Aniso triazine | 0.5 | 0.25 | 1.5 | 0.3 | | 2.0 | 0.75 | 0.5 |
| Octyl triazone | 0.5 | 0.5 | 1.5 | 0.2 | 3.0 | | 0.5 | 1.0 |
| Trisodium EDTA | 0.2 | 0.1 | | 0.05 | | 0.1 | 0.3 | |
| Iminodisuccinate | 0.1 | | 0.1 | | 0.3 | | | 0.5 |
| Phenoxyethanol | 0.3 | 0.1 | | 0.5 | 0.7 | | | 0.4 |
| Parabens | 0.4 | | 0.3 | | 0.3 | | 0.2 | |
| Hexamidine diisethionate | | 0.1 | | | 0.05 | | 0.1 | |
| Imidodiazolidinyl urea | | | | | | 0.2 | | 0.2 |
| DMDM hydantoin | | | 0.2 | | | 0.1 | | |
| Iodopropynyl | | | | 0.2 | | | 0.05 | |
| butylcarbamate | | | | | | | | |
| Glycerin | 10.0 | 3.0 | 7.0 | 8.0 | 15.0 | 20.0 | 0.5 | 2.0 |
| Tocopheryl acetate | 0.2 | 0.5 | 0.75 | | | 0.3 | | 1.0 |
| Alcohol denat. | 5.0 | | 2.5 | | | 7.5 | | 7.5 |
| Water | ad | ad | ad | ad | ad | ad | ad | ad |
| | 100 | 100 | 100 | 100 | 100 | 100 | 100 | 100 |

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A cosmetic preparation which comprises (a) one or more 1,2-alkanediols and (b) one or more UV light-protection filters from the group of triazine derivatives.

2. The preparation of claim 1, wherein the preparation comprises (a) in a total concentration of from about 0.3% to about 3% by weight, based on a total weight of the preparation.

3. The preparation of claim 2, wherein the preparation comprises (a) in a total concentration of from about 0.5% to about 2% by weight.

4. The preparation of claim 1, wherein the preparation comprises (b) in a total concentration of from about 0.3% to about 3% by weight, based on a total weight of the preparation.

5. The preparation of claim 4, wherein the preparation comprises (b) in a total concentration of from about 0.5% to about 2% by weight.

6. The preparation of claim 1, wherein a weight ratio (a): (b) is from about 2:1 to about 1:2.

7. The preparation of claim 6, wherein a weight ratio (a): (b) is about 1:1.

8. The preparation of claim 1, wherein (a) comprises 1,2-hexanediol.

9. The preparation of claim 1, wherein (b) comprises one or more of 2,4-bis{[4-(2-ethylhexyloxy)-2-hydroxy]phenyl}-6-(4-methoxyphenyl)-1,3,5-triazine, dioctylbutylamidotriazone, 2,4-bis-[5-1 (dimethylpropyl)benzoxazol-2-yl-(4-phenyl)-imino]-6-(2-ethylhexyl)-imino-1,3,5-triazine, tris(2-ethylhexyl) 4,4′,4″-(1,3,5-triazine-2,4,6-triyltriimino)trisbenzoate and 2,4,6-tribiphenyl-4-yl-1,3,5-triazine.

10. The preparation of claim 9, wherein (b) comprises one or more of 2,4-bis{[4-(2-ethylhexyloxy)-2-hydroxy]phenyl}-6-(4-methoxyphenyl)-1,3,5-triazine, dioctylbutylamidotriazone, 2,4-bis-[5-1 (dimethylpropyl)benzoxazol-2-yl-(4-phenyl)-imino]-6-(2-ethylhexyl)-imino-1,3,5-triazine and 2,4,6-tribiphenyl-4-yl-1,3,5-triazine.

11. The preparation of claim 1, wherein the preparation comprises one or more additional UV light-protection filters which are different from a triazine derivative.

12. The preparation of claim 11, wherein the one or more additional UV light-protection filters comprise one or more of phenylene-1,4-bis(2-benzimidazyl)-3,3′-5,5′-tetrasulfonic acid salts, 2-phenylbenzimidazole-5-sulfonic acid salts, 1,4-di(2-oxo-10-sulfo-3-bornylidenemethyl)-benzene and salts thereof, 4-(2-oxo-3-bornylidenemethyl)benzenesulfonic acid salts, 2-methyl-5-(2-oxo-3-bornylidenemethyl)sulfonic acid salts, 2,2′-methylenebis[6-(2H-benzotriazol-2-yl)-4-(1,1,3,3-tetramethylbutyl)phenol], 2-(2H-benzotriazol-2-yl)-4-methyl-6-[2-methyl-3-[1,3,3,3-tetramethyl-1-[(trimethylsilyl)oxy]-disiloxanyl]propyl]-phenol, 3-(4-metylbenzylidene)camphor, 3-benzylidene camphor, (2-ethylhexyl) 4-(dimethylamino)benzoate, amyl 4-(dimethylamino)benzoate; di(2-ethylhexyl) 4-methoxybenzalmalonate; dioctylbutylamidotriazone, (2-ethylhexyl) 4-methoxycinnamate, isopentyl 4-methoxycinnamate; 2-hydroxy-4-methoxybenzophenone, 2-hydroxy-4-methoxy-4′-methylbenzophenone, 2,2′-dihydroxy-4-methoxy-benzophenone, hexyl 2-(4′-diethylamino-2′-hydroxybenzoyl)-benzoate, 4-(tert.-butyl)-4′-methoxydibenzoylmethane, homomenthyl salicylate, 2-ethylhexyl-2-hydroxybenzoate, 2-ethylhexyl-2-cyano-3,3-diphenylacrylate, 3-(4-(2,2-bis-ethoxycarbonylvinyl)-phenoxy)propenyl)-methoxysiloxane/-dimethyl siloxane-copolymer, titanium dioxide and zinc oxide.

13. The preparation of claim 1, wherein the preparation further comprises tocopheryl acetate.

14. The preparation of claim 1, wherein the preparation further comprises glycerol.

15. A cosmetic preparation which comprises (a) from about 0.5% to about 2% by weight of one or more 1,2-alkanediols which comprise 1,2-hexanediol and (b) from about 0.5% to about 2% by weight of one or more UV light-protection filters from the group of triazine derivatives, each based on a total weight of the preparation.

16. The preparation of claim 15, wherein a weight ratio (a): (b) is from about 2:1 to about 1:2.

17. The preparation of claim 16, wherein (b) comprises one or more of 2,4-bis{[4-(2-ethylhexyloxy)-2-hydroxy]phenyl}-6-(4-methoxyphenyl)-1,3,5-triazine, dioctylbutylamidotriazone, 2,4-bis-[5-1 (dimethylpropyl)benzoxazol-2-yl-(4-phenyl)-imino]-6-(2-ethylhexyl)-imino-1,3,5-triazine and 2,4,6-tribiphenyl-4-yl-1,3,5-triazine.

18. The preparation of claim 16, wherein the preparation comprises one or more additional UV light-protection filters which are different from a triazine derivative.

19. An emulsion which comprises the preparation of claim 1.

20. A method of improving the solubility of a UV light-protection filter which is a triazine derivative, wherein the method comprises combining the UV light-protection filter with one or more 1,2-alkanediols.
